(19) United States
(12) Patent Application Publication
(10) Pub. No.: US 2021/0307332 A1
(43) Pub. Date: Oct. 7, 2021

(54) POLYMER-STABILIZED AQUEOUS HYDROGEN PEROXIDE SOLUTIONS AND ASSOCIATED METHODS

(71) Applicant: Evonik Corporation, Parsippany, NJ (US)

(72) Inventor: Gregory Melenkevitz, Union, NJ (US)

(73) Assignee: Evonik Corporation, Parsippany, NJ (US)

(21) Appl. No.: 15/733,904

(22) PCT Filed: Aug. 1, 2019
(86) PCT No.: PCT/US2019/044654

§ 371 Date: Dec. 1, 2020

Related U.S. Application Data
(60) Provisional application No. 62/713,790, filed on Aug. 2, 2018.

(51) Int. Cl. A01N 59/00; A01N 25/22; A01N 25/06; A01N 25/04; C01B 15/037; A61L 2/18; D21C 9/16; B65B 55/10

(52) U.S. Cl. CPC A01N 59/00; A01N 25/22; A01N 25/06; A01N 25/04; A61L 2202/23; A61L 2/186; D21C 9/163; B65B 55/10; A61L 2202/18; C01B 15/037

(57) ABSTRACT

Aqueous solutions of hydrogen peroxide are stabilized by at least one polymeric stabilizer selected from phosphino polycarboxylic acids, poly(acrylic acid)-acrylamidoalkylpropane sulfonic acid co-polymers and poly(acrylic acid)-acrylamidoalkylpropane sulfonic acid-sulfonated styrene terpolymers. The polymer-stabilized hydrogen peroxide solutions have applications in aseptic packaging, electronics manufacture, and pulp and paper bleaching.

### CROSS-REFERENCE TO RELATED APPLICATIONS

This application is a 35 U.S.C. § 371 U.S. national phase entry of International Application No. PCT/US2019/044654 having an international filing date of Aug. 1, 2019, which claims the benefit of U.S. Provisional Application No. 62/713,790 filed Aug. 2, 2018, both of which are incorporated herein by reference in its entirety.

### FIELD

The present invention relates to polymer-stabilized aqueous hydrogen peroxide solutions and their use in aseptic packaging, electronics, and pulp and paper bleaching.

### BACKGROUND

Hydrogen peroxide has a variety of industrial uses, as summarized in Table 1.

TABLE 1

| Industry | Application |
|---|---|
| Pulp and paper | Bleaching wood pulp |
| Mining | Detoxification of cyanide tailings |
| Textile bleaching | Bleaching of cotton fabrics |
| Wool scouring | Bleaching of wool |
| Waste water treatment | Measuring dissolved oxygen. Destroying |
| | soluble cyanides, sulfides, and phenols. |
| Packaging | Aseptic packaging of milk and fruit juice |

a. Pulp and Paper

Bleaching of lignocellulosic materials can be divided into lignin retaining and lignin removing bleaching operations. In the case of bleaching high yield pulps like Groundwood, Thermo-Mechanical Pulp and Semi-Chemical pulps, the objective is to brighten the pulp while all pulp components including lignin are retained as much as possible. This kind of bleaching is lignin retaining. Common lignin retaining bleaching agents used in the industry are alkaline hydrogen peroxide and sodium dithionite (hydrosulfite).

In order to reduce energy consumption and improve pulp quality in mechanical pulping, chemical treatments of various types may be employed. These treatments are mild in comparison to those used in the chemical pulping and bleaching. They give “chemically modified” pulps.

The aim is to retain the high yield range of 90-95%, which is a major advantage of mechanical pulping. More severe chemical treatments, which lower the yield to the 85-90% range, are called “chemi-mechanical” pulps. There are three approaches to treatment: pre-treatment, post-treatment, and inter-stage treatment. Pre-treatments of wood chips aim primarily to lower energy consumption. Post-treatments aim to flexibilize fibres, to produce better bonding in paper. Inter-stage treatments aim at some combination of these two. Sulphonation is one common form of chemical treatment. Here wood or fibres are reacted with sodium sulphite or sodium bisulphate to produce a reaction in which sulphonic acid breaks down the lignin in the wood structure. This replaces some lignin groups with sulphite ions. One treatment, a chip pre-treatment for TMP is called “Chemi-thermomechanical” (CTMP) pulping. CTMP fibres are even more flexible and longer than TMP and can result in very strong pulp.

In the case of chemical pulps like kraft pulp, sulfite pulps, NSSC, NSSC-AQ, soda, organosolv, and the like, lignocellulosic material has been subjected to delignifying treatments. Pulping dissolves 85% to 95% of the lignin in the feedstock material. Following the pulping stage, the pulp is washed with water to remove dissolved lignin. While pulping removes most of the lignin in the feedstock material, it is not capable of removing all the lignin without destroying the cellulose fibers of the feedstock. The remaining lignin is removed from the pulp by bleaching.

Bleaching of chemical pulps includes further lignin reducing (delignifying) reactions and is performed in one or more subsequent stages. In bleaching chemical pulp, the initial stages are generally considered as the “delignification stages”. The subsequent stages are called the “final bleaching”. This terminology describes the main effects that can be seen by the specific chemical treatments. While in the initial stages the most apparent effect is the reduction of residual lignin, in the subsequent stages the most distinguishable effect is the increased brightness.

After delignification is usually chemical bleaching with oxidative chemicals, such as chlorine dioxide (ClO₂). However, several processes have been described which may bleach, facilitate bleaching, or enhance bleaching of pulp prior to bleaching with ClO₂. These include (1) the use of hydrogen peroxide and peracids, and (2) the use xylanase enzyme treatment.

A pulp bleaching process may comprise an alkaline oxygen delignification stage (0), an enzymatic treatment stage (X), one or more chlorine dioxide stages (D), and one or more alkaline extraction stages (E). A pulp bleaching process may also comprise one or more water washes or alternatively, each stage may comprise a water wash as a final step of the stage. Thus, a representative pulp bleaching sequence in which pulp is bleached using three chlorine dioxide stages and two alkaline extraction stages may be represented as D-E-D-E-D. Similarly, a pulp bleaching sequence wherein pulp is subjected to an alkaline oxygen delignification stage, an enzymatic treatment stage, three chlorine dioxide bleaching stages and two alkaline extraction stages wherein each stage is followed by a water wash may be represented by O-X-D-E-D-E-D.

Solutions containing only hydrogen peroxide are relatively ineffective in bleaching, and therefore, it is essential to activate them by the addition of alkalis in order to improve the bleaching power. Sodium hydroxide is frequently used to this end. However, if the alkaline agent is added alone, it induces much too rapid and much too great a decomposition of the hydrogen peroxide, so that a not insignificant part of the latter is lost for bleaching. Hydrogen peroxide decomposes into oxygen and water with increasing pH, temperature, heavy metal concentrations, etc. The decomposition products, radicals like HO. and HOO., lead to lower yields by oxidation and degradation of lignin and polyoses. Therefore, hydrogen peroxide is stabilized with sodium silicates and chelating agents when mechanical pulps (high yield pulps) are bleached.

Pulp mills can experience considerable scale deposit problems. Forces that drive inorganic salts to precipitate from pulping and bleaching liquors include pH and temperature shocks, intense mechanical or hydrodynamic shear forces and super-saturation concentrations of scaling ions.

Acid and alkaline bleaching and washing stages in a bleach plant create extreme pH swings that provide ideal conditions for scale formation. If an acid washing stage filtrate can be sewered, then many scaling ions are effectively purged from the pulp. Usually, however, the filtrate is reused and sent back to prior bleaching stages. This feeds scaling species back into the pulp. In alkaline washing/extraction stages, calcium carbonate or oxalate scales are typical. The acid-to-alkaline pH shock and a high concentration of calcium ions are strong driving forces for scale precipitation. Calcium oxalate and/or barium sulfate scales frequently form in chlorine dioxide bleach towers and washers.

Calcium oxalate and barium sulfate scale is a persistent problem in pulp bleaching. Calcium oxalate scale is also a commonly known problem in deinking and sugar processes and has a significant medical and biological importance.

In the pulp bleaching process, the undesirable scale generally deposits on the internal surfaces of the equipment. The scale deposits can inhibit the bleach plant process by, for example, plugging the equipment, such as, the screens, reactors, and internal passages. Chemical deposit control agents are generally known and used to alleviate the scaling problem. These agents act according to three fundamental control mechanisms, that is, inhibition, dispersion, and crystal modification.

There is a need for improved stabilized hydrogen peroxide solutions that allow for reduced amounts of traditional stabilizers or that keep such stabilizers dispersed, thereby reducing precipitation/incrustation.

b. Aseptic Packaging

Chemical sterilization of packaging materials currently makes it possible to make foodstuffs such as milk, yoghurt or fruit juices available to the end user in simple, user-friendly packaging, without treating or impairing the respective foodstuff itself in any way. The high degree of acceptance of such user-friendly packaging results in the filling capacity of the filling machines constantly being increased, which simultaneously is often accompanied by shortening of the filling cycles.

In the chemical sterilization of packaging materials, the chemicals which can be used are limited by food regulations. Only those chemicals or mixtures which are permitted on their own or—in the case of mixtures—the individual constituents of which are permitted under food regulations are permitted to be used.

It has been shown in the past that hydrogen peroxide, as a result of its high oxidizing capacity, is a very effective germicidal medium. Consequently, hydrogen peroxide has now been used successfully for years in almost all aseptic packaging plants in the milk-processing industry and also in juice production etc.

Compared with other germicidal substances or comparable oxidizing agents, hydrogen peroxide has the great advantage of not leaving any residues other than water behind on the packaging materials as a result of the product and of the process, apart from the slight traces of stabilizer.

In the current state of the art of chemical sterilization of packaging materials, essentially two processes have become established on the market, the dip bath process and the spray process. In both these processes, hydrogen peroxide is used as a germicidal agent at elevated temperatures. The demands made on the material-specific properties of the hydrogen peroxide depend on the process in question.

Thus, for example, in the spray process the hydrogen peroxide used should for process-related reasons contain only few inert materials, which very largely originate from the stabilizers used because in the spray process the inert materials result in incrustations in the evaporator or spraying section, which necessitates cleaning and ultimately reduces the filling capacity of the system.

In the dip bath process the germicidal process takes place in a bath filled with hydrogen peroxide. For this, the packaging material is passed through a temperature-controlled bath and during the latter course of the process is mechanically separated from adhering hydrogen peroxide residues. As a result of the process, therefore, the hydrogen peroxide used must be more highly stabilized than the product used in the spray process referred to above. In order to extend the useful life of the hydrogen peroxide used, foodstuff-compatible stabilizers are added to the hydrogen peroxide. It is for example known to use pyrophosphates/phosphoric acid in combination with stannates for stabilization.

There is a need for improved stabilized hydrogen peroxide solutions that allow for reduced amounts of traditional stabilizers or that keep such stabilizers dispersed, thereby reducing precipitation/incrustation.

### SUMMARY

The invention provides improved stability of electronic, aseptic and standard grades of aqueous hydrogen peroxide and especially solutions lightly stabilized with traditional stabilizers. The aqueous hydrogen peroxide solutions allow lower levels of traditional stabilizers in aseptic packing applications and prevent plugging of nozzles in aseptic spray machines. However, any level of typical hydrogen peroxide stabilizer (stannate, phosphate, chelant) may be used with the polymer-stabilized hydrogen peroxide solutions of the invention. The polymeric stabilizers keep inorganic stabilizers dispersed, prevent precipitation, and passivate metal surfaces thereby preventing inorganic deposits from fouling heating elements or heat exchangers. The polymer-stabilized H₂O₂ solutions of the invention allow plants to run longer without the need to shut down for cleaning of heating elements. Thus, the polymeric stabilizer can be used to replace chelants that are typically used for peroxide stabilization as the polymeric stabilizers control trace metals that attack hydrogen peroxide and cause decomposition. Sodium acid pyrophosphate is often used in the manufacturing process of hydrogen peroxide to stabilize the hydrogen peroxide solution prior to it being concentrated. By controlling trace metal contamination, less inorganic phosphate stabilizer can be used reducing the sodium content in the finished peroxide.

The invention provides improved stability of hydrogen peroxide solutions as well as scale control. The use of the polymeric stabilizer will eliminate scaling in many applications where hydrogen peroxide is added which will greatly reduce down time associated with chemical cleaning of equipment. The new stabilizer allows any level of typical hydrogen peroxide stabilizers (stannate, phosphate, chelant) to be used without precipitation/scale from forming and fouling process equipment. The new invention results in eliminating scale where the polymeric stabilizer is used due to the material being added where the chemical reaction is taking place. The invention has particular application in pulp and paper mills for preventing scale on extraction stage washer wires/pump impellers, BCTMP mills (bleached chemi-thermomechanical pulp mills), and recycle mills (pump impellers, disperger plates).

In one aspect, the invention provides an aqueous composition comprising hydrogen peroxide; and one or more polymeric stabilizers selected from

a) a phosphino polycarboxylic acid, or salt thereof, the phosphino polycarboxylic acid having a molecular weight of 1500 to 10,000 g/mol; and

b) a polymer, or salt thereof, with molecular weight of 3000 to 15,000 g/mol, the polymer being derived from a plurality of monomer units of each of

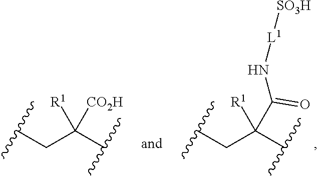

and optionally

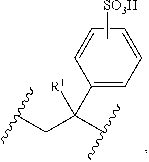

wherein R¹, at each occurrence, is independently hydrogen or C₁₋₄alkyl and L¹ is C₂₋₆alkylene.

In another aspect, the invention provides a process of aseptic sterilization of packaging material comprising dipping the packaging material in or spraying the packaging material with the aqueous composition of the invention.

In another aspect, the invention provides a process of bleaching paper pulp or cellulosic fibers comprising contacting the composition of the invention with the paper pulp or the cellulosic fibers.

### DETAILED DESCRIPTION OF THE INVENTION

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present invention. All publications, patent applications, patents and other references mentioned herein are incorporated by reference in their entirety. The materials, methods, and examples disclosed herein are illustrative only and not intended to be limiting.

For the recitation of numeric ranges herein, each intervening number there between with the same degree of precision is explicitly contemplated. For example, for the range 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9 and 7.0 are explicitly contemplated.

The modifier “about” used in connection with a quantity is inclusive of the stated value and has the meaning dictated by the context (for example, it includes at least the degree of error associated with the measurement of the particular quantity). The modifier “about” should also be considered as disclosing the range defined by the absolute values of the two endpoints. For example, the expression “from about 2 to about 4” also discloses the range “from 2 to 4.” The term “about” may refer to plus or minus 10% of the indicated number. For example, “about 10%” may indicate a range of 9% to 11%, and “about 1” may mean from 0.9-1.1. Concentrations and fractions given in “%” and “ppm” refer to weight unless specified otherwise.

Compositions

Aqueous hydrogen peroxide solutions may be produced by the anthraquinone process. A survey of the anthraquinone process and its numerous modifications is given in G. Goor, J. Glenneberg, S. Jacobi: “Hydrogen Peroxide” Ullmann's Encyclopedia of Industrial Chemistry, Electronic Release, 6th ed. Wiley-VCH, Weinheim June 2000, page 14. Generally, the anthraquinone loop process comprises the following steps:
(a) Hydrogenation of a working solution comprising an organic solvent or mixture of organic solvents, and one or more active anthraquinone compounds;
(b) oxidation of the hydrogenated working solution to form hydrogen peroxide;
(c) extraction of hydrogen peroxide with water;
(d) stabilizing of the extracted aqueous hydrogen peroxide solution;
(e) drying of the working solution after extraction; and
(f) regeneration and purification of the working solution.

Crude hydrogen peroxide solutions or concentrated hydrogen peroxide solutions obtained from the anthraquinone process typically contain a plurality of compounds in addition to hydrogen peroxide in low concentrations. These compounds are either impurities or additives like stabilizers. The impurities are compounds that are extracted from the working solution into the aqueous phase. They are mainly ionic or polar species like carboxylic acids, alcohols, carbonyl compounds and amines. These impurities are therefore also found in some commercial hydrogen peroxide solutions.

For example, hydroquinone solvents that are commonly used in the above described process are nitrogen containing compounds like amides and ureas (see Ullmann supra page 6). Examples include tetraalkyl ureas like tetrabutyl urea. The use of these solvents result in amine impurities like monoalkyl or dialkyl especially monobutyl and dibutyl amines in the final hydrogen peroxide solutions. For example, some commercial hydrogen peroxide solutions may contain up to 200 ppm mono- and dibutyl amine based on the weight of hydrogen peroxide.

Thus, aqueous hydrogen peroxide solutions prepared by the anthraquinone process may contain organic impurities (products of degradation of the quinone shuttle, traces of diluent) and inorganic impurities (cations and anions introduced by the extraction water, as well as those already present in the mixture derived from the oxidation of the alkylanthraquinone(s)).

Aqueous hydrogen peroxide solution may thus comprise organic impurities expressed as TOC (total organic carbon concentration), defined according to ISO standard 8245. The TOC may contain organic compounds such as, for example, dimethyheptanol (DMH), diisobutylcarbinol (DiBC), 2,6-dimethyl-1,4-heptanediol (C₉H₂₀O₂), methyl cyclohexyl acetate, methyl cyclohexanol, tetrabutyl urea (TBU), trioctylphosphate (TOP), and/or degradation products of alkylated aromatic solvents such as Solvesso 150, i.e. corresponding to the product compounds oxidized on their alkyl chain. The TOC may contain DiBC, methyl cyclohexyl acetate, TBU and/or TOP in an amount of from 30 to 200 ppm by weight of solution, from 50 to 150 ppm, an amount of about 100 ppm being common.

Depending on the final use of the hydrogen peroxide solutions, purification steps may be conducted in order to obtain the required specification for the respective use of the hydrogen peroxide solution. For example, food and electronics grade hydrogen peroxide solutions require higher purity levels than solutions intended for use in pulp and paper bleaching. U.S. Pat. No. 6,939,527 discloses a purification process for aqueous hydrogen peroxide solutions, whereby the solutions are treated with an anion exchange resin, a nonionic absorbing resin having a specific structure, and a neutral absorbing resin also having a specific macroporous structure. The hydrogen peroxide solutions obtained in this way are substantially free of cationic, anionic and organic impurities. Therefore, the solutions are particularly useful⋅in microelectronics applications. Similarly U.S. Pat. No. 4,999,179 discloses a process for purification of hydrogen peroxide solutions that contain after purification each metal cation in an amount of less than 5 ppb, each anion in an amount of less than 10 ppb and organic impurities in an amount of not more than 5 ppm in terms of total organic carbon content.

In one embodiment, the aqueous hydrogen peroxide solution of the invention has been subjected to at least one subsequent purification step. The subsequent purification step can consist of any method which is well known to those skilled in the art for reducing the impurity content of an aqueous hydrogen peroxide solution. A type of purification step which can be employed is a washing operation with at least one organic solvent, as the one described in European patent application EP 0965562. This document is incorporated herein by reference. Other purification techniques include reverse osmosis, microfiltration, ultrafiltration, nanofiltration, ion exchange resin treatment, nonionic absorber resin treatment, and neutral absorber resin treatment, as described in U.S. Pat. Nos. 8,715,613, 6,333,018, 5,215,665, 5,232,680, 6,939,527, 4,999,179, 4,879,043, 3,297,404, 3,043,666, EP552187, EP0930269, WO2005/033005, and Abejon et al., Separation and Purification Technology (2010) 76, 44-51, which are hereby incorporated by reference.

Microfiltration (MF) removes particles in the range of approximately 0.1-1 μm. In general, suspended particles and large colloids are rejected while macromolecules and dissolved solids pass through the MF membrane. Applications include removal of bacteria, flocculated materials, or TSS (total suspended solids). Transmembrane pressures are typically 10 psi (0.7 bar).

Ultrafiltration (UF) provides macro-molecular separation for particles ranging in size from approximately 20-1,000 Angstroms (up to 0.1 μm). All dissolved salts and smaller molecules pass through the membrane. Items rejected by the membrane include colloids, proteins, microbiological contaminants, and large organic molecules. Most UF membranes have molecular weight cut-off values between 1,000 and 100,000 g/mol. Transmembrane pressures are typically 15-100 psi (1-7 bar).

Nanofiltration (NF) refers to a membrane process which rejects particles in the approximate size range of 1 nanometer (10 Angstroms), hence the term “nanofiltration.” NF operates in the realm between UF and reverse osmosis. Organic molecules with molecular weights greater than 200-400 g/mol are rejected. Also, dissolved salts are rejected in the range of 20-98%. Salts which have monovalent anions (e.g., sodium chloride or calcium chloride) have rejections of 20-80%, whereas salts with divalent anions (e.g., magnesium sulfate) have higher rejections of 90-98%. Typical applications include removal of color and total organic carbon (TOC) from surface water, removal of hardness or radium from well water, overall reduction of total dissolved solids (TDS), and the separation of organic from inorganic matter in specialty food and wastewater applications. Transmembrane pressures are typically 50-225 psi (3.5-16 bar).

Reverse osmosis (RO) membranes generally act as a barrier to all dissolved salts and inorganic molecules, as well as organic molecules with a molecular weight greater than approximately 100 g/mol. Water molecules, on the other hand, pass freely through the membrane creating a purified product stream. Rejection of dissolved salts is typically 95% to greater than 99%, depending on factors such as membrane type, feed composition, temperature, and system design.

Aqueous hydrogen peroxide solutions may be subjected to one or more of the foregoing purification techniques or sequentially subjected to the same purification technique more than once to achieve higher levels of purity. For example, for food grade hydrogen peroxide solutions, reverse osmosis purification may be carried out at least once (e.g., 1-2 times). For electronics grade hydrogen peroxide solutions reverse osmosis may be carried out at least twice (e.g., 2-3 times). Standard grade hydrogen peroxide refers to hydrogen peroxide solutions having higher concentrations of residue upon evaporation and that would not be suitable for food or electronics applications. In some embodiments, standard grade solutions have not undergone treatment by techniques such as reverse osmosis. In some embodiments, standard grade hydrogen peroxide is a solution remaining that did not pass a reverse osmosis membrane.

The polymer-stabilized aqueous hydrogen peroxide solution according to the invention generally has a hydrogen peroxide concentration [H₂O₂] expressed as % by weight of the solution. The crude hydrogen peroxide may be vacuum distilled to concentrations of up to 70% w/w. The hydrogen peroxide solution may be concentrated to a hydrogen peroxide concentration of at least 50% by weight, at least 60% by weight, or from 60 to 70% by weight, based on the total weight of the hydrogen peroxide solution. Alternatively, the hydrogen peroxide concentration may be 80% or less, 75% or less, or 60% or less. Depending on the application, the hydrogen peroxide concentration [H₂O₂] may be at least 5%, in particular at least 10%, in many cases equal to or more than 20%, or equal to or even more than 30%. Concentrations of at least 32%, at least 35%, at least 38%, are usual. For example, hydrogen peroxide concentrations of around 40% or 50% are common.

In aspetic packaging applications, H₂O₂ concentrations are typically about 35%. For example, the hydrogen peroxide concentration may be 35.0 to 36.0% or 34.0 to 34.9%. Hydrogen peroxide concentrations used for pulp and paper bleaching are typically lower, e.g., about 0.1-5%. In the case of bleaching kraft pulp, the concentration may be around 0.1-1%. In the case of a chemi-thermomechanical pulp, the concentration may be around 1-5%. 50-70% aqueous H₂O₂ solutions produced according to the disclosed methods may be diluted to appropriate concentrations according to the particular use.

In some embodiments, the polymer-stabilized aqueous hydrogen peroxide solution of the invention is prepared by adding the one or more polymeric stabilizers to an aqueous hydrogen peroxide solution that has been subjected to a purification technique (e.g., reverse osmosis) to reduce the levels of TOC and metals/inorganics. A polymeric stabilizer may be added earlier in the anthraquinone process, for example, after extraction and/or before concentration or other purification. Adding a polymeric stabilizer after purification, however, can replace any polymeric stabilizer lost through the purification process (e.g., reverse osmosis).

In some embodiments, the one or more polymeric stabilizers are selected from a phosphino polycarboxylic acid, or salt thereof. The phosphino polycarboxylic acid has formula (I)

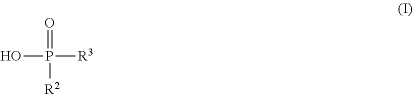

wherein R² is

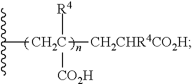

### R³ is

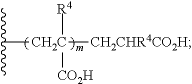

R⁴, at each occurrence, is independently hydrogen or C₁₋₄alkyl; and m and n are each independently an integer, where m+n is an integer from 30 to 60. In some embodiments, R⁴ is hydrogen. In some embodiments, the phosphino polycarboxylic acid has a molecular weight of 3300-3900 g/mol.

In some embodiments, the one or more polymeric stabilizers is selected from a poly(acrylic acid), or a salt thereof. In some embodiments, the poly(acrylic acid), or salt thereof, has a molecular weight of 4100-4900 g/mol.

In some embodiments, the one or more polymeric stabilizers is selected from a polymer, or salt thereof, with molecular weight of 3000 to 15,000 g/mol, the polymer being derived from a plurality of monomer units of each of

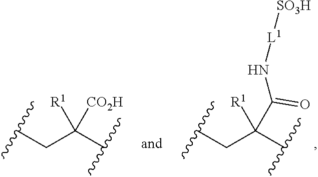

wherein R¹, at each occurrence, is independently hydrogen or C₁₋₄alkyl and L¹ is C₂₋₆alkylene. In some embodiments, the polymer is derived from a plurality of monomer units of each of

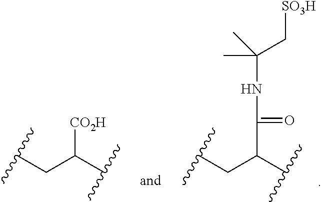

The polymeric stabilizers preferably consist of the specified monomer units. In some embodiments, the one or more polymeric stabilizers is selected from a polymer, or salt thereof, with molecular weight of 3000 to 15,000 g/mol, the polymer being derived from a plurality of monomer units of each of

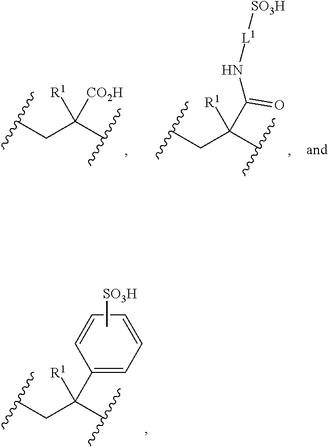

wherein R¹, at each occurrence, is independently hydrogen or C₁₋₄alkyl and L¹ is C₂₋₆alkylene. In some embodiments, the polymer is derived from a plurality of monomer units of each of

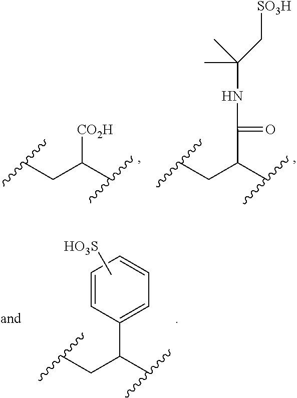

The polymeric stabilizers preferably consist of the specified monomer units.

Unless otherwise specified, as used herein a polymer molecular weight refers to a weight average molecular weight of a polymer sample measured by gel permeation chromatography (GPC).

In some embodiments, the salt of a polymeric stabilizer is an alkali metal salt. In some embodiments, the alkali metal salt is a sodium salt.

The term “alkyl” as used herein, means a straight or branched chain saturated hydrocarbon. Representative examples of alkyl include, but are not limited to, methyl, ethyl, npropyl, isopropyl, n-butyl, sec-butyl, isobutyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, and n-decyl.

The term “alkylene,” as used herein, means a divalent group derived from a straight or branched chain saturated hydrocarbon. Representative examples of alkylene include, but are not limited to, —CH₂—, —CH₂CH₂—, —CH₂CH₂CH₂—, —CH₂CH(CH₃)CH₂—, and CH₂CH(CH₃)CH(CH₃)CH₂—.

Terms such as “alkyl” and “alkylene,” may be preceded by a designation indicating the number of atoms present in the group in a particular instance (e.g., “C₁₋₄alkyl,” “C₁₋₄alkylene”). These designations are used as generally understood by those skilled in the art. For example, the representation “C” followed by a subscripted number indicates the number of carbon atoms present in the group that follows. Thus, “C₃alkyl” is an alkyl group with three carbon atoms (i.e., n-propyl, isopropyl). Where a range is given, as in “C₁₋₄,” the members of the group that follows may have any number of carbon atoms falling within the recited range. A “C₁₋₄alkyl,” for example, is an alkyl group having from 1 to 4 carbon atoms, however arranged (i.e., straight chain or branched).

The polymeric stabilizers may be added to the about 25-40% H₂O₂ solution obtained from extraction and prior to concentration in an amount suitable to prevent scale formation during concentration. In some embodiments, the extracted hydrogen peroxide solution is stabilized with at least 0.1-1500 ppm of the one or more polymeric stabilizers. In some embodiments, the peroxide solution is stabilized with from 0.1-60 ppm, 0.1-50 ppm, 0.1-40 ppm, 0.1-30 ppm, 0.1-20 ppm, 0.1-10 ppm, 10-20 ppm, 20-30 ppm, 30-40 ppm, 40-50 ppm, or 50-60 ppm of the one or more polymeric stabilizers. In other embodiments, the peroxide solution is stabilized with higher concentrations of the one or more polymeric stabilizers. For example, the 25-40% hydrogen peroxide solution may be stabilized with from 50-150 ppm, 150-250 ppm, 250-350 ppm, 350-650 ppm, 600-900 ppm, 800-1200 ppm, or 1200-1600 ppm of the one or more polymeric stabilizers. In some embodiments, the one or more polymeric stabilizers are added in an amount ≥100 ppm, ≥200 ppm, ≥300 ppm, ≥500 ppm, ≥750 ppm, ≥1000 ppm, ≥1500 ppm, or ≥2000 ppm.

Levels of polymeric stabilizer ≤60 ppm are suited for aseptic packaging applications with about 35% H₂O₂ solutions. Thus, following purification of a crude H₂O₂ solution to a level suitable for aseptic packaging/food applications, polymeric stabilizers may be added in amounts that would provide ≤60 ppm polymeric stabilizer in an about 35% H₂O₂ solution. For example, a purified 70% H₂O₂ solution may be stabilized with ≤120 ppm of polymeric stabilizer for eventual twofold dilution of H₂O₂ prior to the end use. In some embodiments, a purified H₂O₂ solution is stabilized with amounts of polymeric stabilizer(s) that provides 0.1-60 ppm, 0.1-50 ppm, 0.1-40 ppm, 0.1-30 ppm, 0.1-20 ppm, 0.1-10 ppm, 10-20 ppm, 20-30 ppm, 30-40 ppm, 40-50 ppm, or 50-60 ppm of the one or more polymeric stabilizers in an about 35% H₂O₂ solution.

For concentrated standard grade H₂O₂ solutions not subjected to high level purification, additional polymeric stabilizer may be added in amounts suitable for the particular end use. In some embodiments, a standard grade hydrogen peroxide solution is stabilized with higher concentrations of the one or more polymeric stabilizers. For example, a 50% hydrogen peroxide solution may be stabilized with from 50-150 ppm, 150-250 ppm, 250-350 ppm, 350-650 ppm, 600-900 ppm, 800-1200 ppm, or 1200-1600 ppm of the one or more polymeric stabilizers. In some embodiments, the one or more polymeric stabilizers are added in an amount ≥100 ppm, ≥200 ppm, ≥300 ppm, ≥500 ppm, ≥750 ppm, ≥1000 ppm, ≥1500 ppm, or ≥2000 ppm. Higher amounts of polymeric stabilizers in a 50% standard grade hydrogen peroxide may have downstream applications in pulp and paper bleaching, bearing in mind the expected dilutions under bleaching conditions in the mill. Additional polymeric stabilizer may be added as needed prior to bleaching.

For more concentrated hydrogen peroxide solutions, polymeric stabilizer amounts may increase proportionately relative to the amounts present in a 35% hydrogen peroxide solution. In some embodiments, the polymeric stabilizer concentrations for a Y % H₂O₂ solution may be determined according to an equation:

$$stabilizer ppm ( Y \% ) = \frac{Y \% H 2 O 2}{35 \% H 2 O 2} \times stabilizer ppm ( 35 \% )$$

For example, a 70% H₂O₂ solution may nave a polymeric stabilizer concentration twice that of a 35% solution.

The use of the polymeric stabilizer system herein does not preclude or restrict the presence of other known stabilizers. Stabilized solutions of the invention may include additional stabilizers or additives, such as a phosphate, a stannate, a chelant, or a radical scavenger. Stabilizers may also be chosen from nitric acid, phosphoric acid, benzoic acid, dipicolinic acid (DPA), from salts chosen from nitrate, phosphate, pyrophosphate, stannate, benzoate, salicylate, diethylene triamine penta (methylene phosphonate), and mixtures thereof. The salts may be ammonium or alkaline metal salts, especially ammonium or sodium salts. The stabilizer may be chosen from nitric acid, phosphoric acid, di-sodium pyrophosphate, ammonium nitrate, sodium nitrate, sodium stannate, and mixtures thereof. The stabilizer may be added in amount of from 0.1 to 200 ppm, 0.1 to 100 ppm, 0.1 to 50 ppm, 0.1 to 40 ppm, 0.1 to 30 ppm, 0.1 to 20 ppm, 0.1 to 10 ppm, 0.1 to 5 ppm. Those amounts are those based on the weight of the solution. In some embodiments, nitric acid is added after reverse osmosis.

Useful stannates include an alkali metal stannate, particularly sodium stannate (Na₂(Sn(OH)₆). Stannates further include stannic chloride, stannic oxide, stannic bromide, stannic chromate, stannic iodide, stannic sulfide, tin dichloride bis(2,4-pentanedionate), tin phthalocyanine dichloride, tin acetate, tin t-butoxide, di-n-butyl tin(IV) dichloride, tin methacrylate, tin fluoride, tin bromide, stannic phosphide, stannous chloride, stannous fluoride, stannous pyrophosphate, sodium stannate, stannous 2-ethylhexoate, stannous bromide, stannous chromate, stannous fluoride, stannous methanesulfonate, stannous oxalate, stannous oxide, stannous sulfate, stannous sulfide, barium stannate, calcium stannate, copper(II) stannate, lead stannate dihydrate, zinc stannate, sodium stannate, potassium stannate trihydrate, strontium stannate, cobalt(II) stannate dihydrate, sodium trifluorostannate, ammonium hexachlorostannate, and lithium hexafluorostannate.

Chelants may be selected from amino tri(methylene phosphonic acid) (ATMP), 2-phosphonobutane-1,2,4-tricarboxylic acid (PBTCA), N-sulfonic amino di(methylene phosphonic acid) (SADP), methylamine d(imethylene phosphonic acid) (MADMP), glycine dimethyl phosphonic acid (GDMP), 2-hydroxyphosphonocarboxylic acid (HPAA), polyhydric alcohol phosphate ester (PAPE), 1-hydroxyethylidene-1,1-diphosphonic acid (HEDP), 1-aminoethane-1,1-diphosphonic acid, ethylene diamine tetra(methylenephosphonic acid), hexamethylene diamine tetra(methylenephosphonic acid), diethylenetriamine penta(methylenephosphonic acid) (DTPMP), diethylenetriamine hexa(methylenephosphonic acid), and 1-aminoalkane-1,1-diphosphonic acids such as morpholinomethane diphosphonic acid, N,N-dimethyl aminodimethyl diphosphonic acid, aminomethyl diphosphonic acid, or a salt thereof.

A phosphate salt can take the form of the simple monomeric species, or of the condensed linear polyphosphate, or cyclic polyphosphate(metaphosphate). The monomeric phosphate salts are of the general formula, MₙH_{q}PO₄, (in which q=0, 1, or 2; n=1, 2, or 3; n+q=3). Here M can be one or more monovalent cations selected from the following: Li, Na, K, NH₄, NR₄ (where R represents an alkyl chain containing 1 to 5 C atoms). The polyphosphates have the general formula, Mₙ₊₂PₙO₃ₙ₊₁ where n=2 to 8, and M can be chosen from Li, Na, K, NH₄, NR₄ where R represents an alkyl chain containing 1 to 5 C atoms). The cyclic polyphosphates have the general formula MnPnO₃n where n=3 to 8 and M can be chosen from Li, Na, K, NH₄, NR₄ where R represents a linear or branched alkyl group containing 1 to 5 C atoms). The above may be optionally introduced into the stabilizer system in their acid form. Exemplary phosphates include pyrophosphoric acid and metaphosphoric acid and their salts, e.g., sodium salts.

Also to be contemplated as phosphorus containing salts are organophosphonates which may be introduced as a soluble salt or as the parent acid. Compounds which may be contemplated include ethylphosphonic acid, propylphosphonic acid, butylphosphonic acid, t-butylphosphonic acid, or phenylphosphonic acid. Additionally the phosphonic acid molecules can contain other functional groups such as hydroxy or amino. These are exemplified in compounds such; as 1-hydroxyethylidene-1,1-diphosphonic acid, and poly(methyleneamino) phosphonic acids such as amino(trimethylene phosphonic acid), and diethylenetriaminepenta(methylenephosphonic acid).

Yet further stabilizers to be contemplated are free radical scavengers. In general, the free radical scavenger may be an organic chelating agent such as a salicylic acid, quinoline, pyridine-2-carboxylic acid, and mixtures thereof. Suitable aromatic chelating agents or aromatic radical scavengers include carbocyclic aromatic rings, such as the benzene or naphthalene ring, as well as heteroaromatic rings such as pyridine and quinoline. The stabilizer may also contain chelating groups, such as hydroxyl, carboxyl, phosphonate, or sulfonate. The aromatic chelating agent may be, for example, a salicylic acid. Any suitable salicylic acid may be used. Salicylic acids may include, for example, a substituted salicylic acid, such as 3-methylsalicylic acid, 4-methyl salicylic acid, 5-methyl salicylic acid, 6-methyl salicylic acid, 3,5-dimethyl salicylic acid, 3-ethyl salicylic acid, 3-iso-propyl salicylic acid, 3-methoxy salicylic acid, 4-methoxy salicylic acid, 5-methyoxy salicylic acid, 6-methoxy salicylic acid, 4-ethoxy salicylic acid, 5-ethyoxy salicylic acid, 2-chloro salicylic acid, 3-chlorosalicylic acid, 4-chloro salicylic acid, 5-chloro salicylic acid, 3,5-dichloro salicylic acid, 4-fluoro salicylic acid, 5-fluoro salicylic acid, 6-fluoro salicylic acid; or a mixture thereof. In a preferred embodiment, the salicylic acid is salicylic acid of the formula C₆H₄(OH)COOH. The aromatic chelating agent may be, for example, 8-hydroxy-quinoline; a substituted 8-hydroxy-quinoline, such as, 5-methyl-8-hydroxyquinoline, 5-methoxy-8-hydroxy-quinoline, 5-chloro-8-hydroxy-quinoline, 5,7-dichloro-8-hydroxy-quinoline, 8-hydroxy-quinoline-5-sulfonic acid, or a mixture thereof. The aromatic chelating agent may be, for example, a pyridine-2-carboxylic acid, such as picolinic acid (2-pyridinecarboxylic acid); dipicolinic acid (2,6-pyridinedicarboxylic acid); 6-hydroxy-picolinic acid; a substituted 6-hydroxy-picolinic acid, such as 3-methyl-6-hydroxy-picolinic acid, 3-methoxy-6-hydroxy-picolinic acid, 3-chloro-6-hydroxy-picolinic acid, or a mixture thereof. Preferred aromatic chelating agents include salicylic acid, 6-hydroxy-picolinic acid, and 8-hydroxy-quinoline. A free radical scavenger may function as both a free radical inhibitor and a chelating agent.

In some embodiments, the polymer-stabilized hydrogen peroxide solutions have a TOC of at most 500 ppm, at most 300 ppm, at most 250 ppm, or at most 100 ppm. Preferably the TOC content is ≤100 ppm for aseptic packing applications.

The aqueous hydrogen peroxide solution may also contain metal cations such as alkali metals or alkaline earth metals, for instance sodium, and/or anions such as phosphates, nitrates, etc. The alkaline and alkaline earth metals may be present in an amount of from 1 to 200 ppm, from 20 to 30 ppm, based on the weight of the solution. The anions (e.g., nitrate) may be present in an amount of from 50 to 500 ppm, or from 100 to 300 ppm based on the weight of the solution. In some embodiments, nitrate may be present in an amount of about 200 ppm.

Generally, phosphate may be present in amount to stabilize any iron present. In the manufacturing process, phosphate may be present in a crude hydrogen peroxide solution of about 40% at about 50-200 ppm. Following concentration to 50-70% hydrogen peroxide, standard grade hydrogen peroxide may have about 200-300 ppm phosphate. In some embodiments, the polymer-stabilized aqueous hydrogen peroxide solution has a phosphorus content expressed as PO₄³⁻ of ≤10 ppm, in some embodiments ≤5 ppm, in some embodiments ≤2 ppm. In some embodiments, the foregoing concentrations refer to solutions with a H₂O₂ concentration of about 35 weight %, where the phosphate concentration will vary proportionately with the H₂O₂ concentration.

The stabilized hydrogen peroxide solutions of the invention may have low levels of transition metals and/or other inorganic components such as antimony, arsenic, cadmium, chromium, copper, iron, lead, nickel, mercury, selenium and tin. The levels of the foregoing may be ≤1 ppm. In some embodiments, tin may be present in an amount of ≤10 ppm. In some embodiments, iron may be present in an amount ≤0.1 ppm. In other embodiments, the following levels may be present: iron ≤0.1 ppm; and arsenic, cadmium, lead, chromium, antimony, mercury, nickel, and selenium ≤1 ppm. In other embodiments, the level of iron is ≤0.05 ppm. In yet other embodiments, the following levels may be present: iron ≤0.05 ppm; arsenic, cadmium, and lead ≤0.02 ppm; chromium ≤0.1 ppm; and antimony, mercury, nickel, and selenium ≤1 ppm. In some embodiments, the foregoing concentrations refer to solutions with a H₂O₂ concentration of about 35 weight %, where the metal concentration will vary proportionately with the H₂O₂ concentration.

In some embodiments, the aqueous hydrogen peroxide solution is free of, or substantially free of, stannate. In some embodiments, the hydrogen peroxide solution is free of, or substantially free of, stannate and/or phosphate.

In some embodiments, the aqueous hydrogen peroxide solution has ≤30, ≤25, ≤20, ≤15, ≤10, ≤5, or ≤1 ppm of a chelating substance other than the one or more polymeric stabilizers. In some embodiments, the aqueous hydrogen peroxide solution is free of, or substantially free of, a chelating substance other than the one or more polymeric stabilizers.

In some embodiments, the aqueous hydrogen peroxide solution consists essentially of hydrogen peroxide, water, and the polymeric stabilizer, as described herein. In other embodiments, the aqueous hydrogen peroxide solution consists essentially of hydrogen peroxide, water, a phosphate, and the polymeric stabilizer, as described herein.

Besides the main ingredients discussed above and any unavoidable impurities in the composition, it is preferred that the balance up to 100% is mainly made up of water.

Sulfur-containing acidifying agents are selected from the group consisting of sulfonic acids, sulfuric acid, alkali metal bisulfates, and mixtures thereof. It will be readily apparent to one of skill in the art that the one or more acidifying agents may be an acid or a salt depending on the pH of the composition. The sulfonic acids may include acids with the general formula R—S(═O)₂—OH, where R may be hydrogen, aliphatic, cyclic, alicyclic or aromatic and the aliphatic part may be a linear or branched, saturated or unsaturated, substituted or unsubstituted hydrocarbon group. In an exemplary embodiment of the present invention, at least one acidifying agent is selected from the group consisting of alkyl sulfonic acids of the formula RSO₃H where R has 10 or fewer carbon atoms; alkyl aryl sulfonic acids of the exemplary formula R¹¹C₆H₄SO₃H where R¹¹ has 7 or fewer carbon atoms; dialkyl aryl sulfonic acids of the formula R²⁰(R³⁰)C₆H₃SO₃H where R²⁰ and R³⁰ together have 7 or fewer carbon atoms; multi-alkyl multi-aromatic-rings-containing sulfonic acid with total 20 or fewer carbon atoms and mixtures thereof, wherein R, R¹¹, R²⁰, and R³⁰ are each individually linear or branched, saturated or unsaturated, substituted or unsubstituted alkyl groups. In one embodiment, at least one acidifying agent is methane sulfonic acid.

Other suitable sulfur-containing acids or salts thereof may include sulfuric acid (H₂SO₄), sulfinic acids, sulfurous acids, bisulfite, bisulfates, etc. Alkali metal bisulfates include alkali metal salts or esters of sulphuric acid containing the monovalent group —HSO₄ or the ion HSO₄—.

In some embodiments, the polymer-stabilized hydrogen peroxide solutions have an acidity as H₂SO₄ of ≤300 ppm, in some embodiments ≤250 ppm, in some embodiments ≤100 ppm, in some embodiments ≤3 ppm.

Phosphoric acid (H₃PO₄) may be used to lower pH and form a relatively stable hydrogen peroxide composition. A stabilized hydrogen peroxide solution of the invention may be entirely phosphate free or free of additional phosphate constituents. Thus, a composition may be termed “phosphate free” even if minor amounts of phosphate are present, for example, as an impurity from the raw materials, but no phosphate, such as phosphoric acid, is intentionally added. In an exemplary embodiment, the hydrogen peroxide composition does not comprise a phosphoric acid or salt thereof (e.g., for use as an acidifying agent, chelating agents, water softener, pH buffering agent, or otherwise).

In some embodiments, after subjecting the aqueous hydrogen peroxide solution to reverse osmosis purification, an about 70% aqueous hydrogen peroxide solution has a residue after evaporation of ≤120 ppm, ≤80 ppm, or ≤40 ppm. Such solutions may be diluted twofold to ≤60, ≤40 or ≤20 ppm for food/aseptic packaging applications with 35% hydrogen peroxide solutions. In some embodiments, an about 35 wt. % aqueous hydrogen peroxide solution suitable for food applications has a residue after evaporation of ≤60 ppm. Solutions with a residue after evaporation of ≤60 ppm are suitable for grades of hydrogen peroxide used for treating/sterilizing packaging materials (e.g. food packaging) using immersion bath techniques. In some embodiments, the aqueous hydrogen peroxide solution has a residue after evaporation of ≤40 ppm. Solutions with a residue after evaporation of ≤40 ppm are suitable for grades of hydrogen peroxide used for treating/sterilizing packaging materials (e.g. food packaging) using spraying techniques or immersion bath techniques. In some embodiments, the aqueous hydrogen peroxide solution has a residue after evaporation of ≤20 ppm. Solutions with a residue after evaporation of ≤20 ppm are suitable for grades of hydrogen peroxide used for treating/sterilizing packaging materials (e.g. food packaging) using spraying techniques. For more concentrated or dilute H₂O₂ solutions, the residue after evaporation will also vary proportionately.

In some embodiments, the retentate after reverse osmosis purification or the aqueous hydrogen peroxide solution prior to purification or concentration may have a higher residue after evaporation of ≥about 800, ≥about 1000, ≥about 1200, ≥about 1400, ≥about 1600, ≥about 1800, or about ≥2000 ppm. Such solutions may be suitable for applications in pulp and paper bleaching.

The residue after evaporation can be determined using the following general procedure:
Clean a platinum dish of suitable size with sea sand by placing a small quantity of the sand into the dish, dampening it and then rubbing it around the dish with a soft cloth so that the surface of the dish is roughened. After each cleaning wash the platinum dish very carefully with distilled water. Add a few milliliters of distilled water to the prepared dish, then place the platinum dish into a larger flat porcelain dish containing distilled water as cooling medium. Smaller platinum dishes can be placed directly into a thermostat at 40° C.
Cover the platinum dish with a watchglass in order to avoid mistakes caused by splashing. Add the hydrogen peroxide in small portions to avoid a violent decomposition. The hydrogen peroxide decomposition samples are usually between 50-200 ml. After decomposition heat the sample using the water bath and after degassing completely remove the watchglass and rinse it off into the platinum dish. The sample is evaporated until almost dry and the residue is rinsed into a quartz glass dish. If only the evaporation residue is to be determined, this can take place directly in the platinum dish. The dish contents must however be rinsed into a quartz glass dish when the residue is to be treated further, because the presence of phosphoric acid or phosphates can damage the platinum dish. Before analysis, boil the quartz glass dish with hydrochloric acid 37% p.a., rub it with sea sand and rinse it with distilled water. Dry the dish at 105° C., calcine it, cool it in a desiccator and finally weigh it. In this dish the sample is evaporated until dryness and then dried in a drying cabinet until a constant weight is reached. After cooling in a desiccator weigh the dish with the residue.
Calculation:

Evaporation residue (mg/l)=residue found (mg)×100/volume of sample (ml)

Evaporation residue (ppm)=residue found (mg/l)/density of sample

The polymer-stabilized hydrogen peroxide solutions described herein have stability at elevated temperature for extended time periods. In some embodiments, after 16 hours at 96° C. the hydrogen peroxide concentration of the aqueous hydrogen peroxide solution is reduced by ≤about 5 weight %. In further embodiments, after 16 hours at 96° C. the hydrogen peroxide concentration of the aqueous hydrogen peroxide solution is reduced by ≤about 3.5 weight %. In still further embodiments, the reduction in hydrogen peroxide concentration is measured in the presence of 0.2 ppm iron, 0.3 ppm aluminum, 0.1 ppm nickel, and/or 0.1 ppm chromium. In some embodiments, the foregoing decomposition results refer to solutions with a H₂O₂ concentration of about 35 weight %. At higher H₂O₂ concentrations, and thus higher polymeric stabilizer concentrations, decomposition amounts are expected to be further reduced.

The polymer-stabilized aqueous hydrogen peroxide solution of the invention generally may have a conductivity of from 20 to 150 μS/cm, for example from 50 to 90 μS/cm. In some embodiments, the conductivity of the stabilized hydrogen peroxide solutions is ≥40 μS/cm. In other embodiments, the conductivity is ≥60 μS/cm. The conductivity of the aqueous solution can be adjusted by the addition therein of a salt, such as for instance ammonium nitrate or mineral acid.

The apparent pH of the aqueous hydrogen peroxide solution according to the invention may be adjusted to the sought value. The pH may be adjusted by any acid, such as by the addition of a sulfur-containing acid, nitric acid and/or phosphoric acid.

In some embodiments, the aqueous hydrogen peroxide solution has a pH≤4. Crude solutions of hydrogen peroxide may have a pH around 3-4. Final product pH is typically around 1-4, depending on the concentration. In some embodiments, the pH is about 1-2, for example with a 70 wt. % hydrogen peroxide solution. In other embodiments, the pH is about 1-3, for example with a 50 wt. % hydrogen peroxide solution. In other embodiments, the pH is 1.5 to 3.5, for example, for a 35 wt. % hydrogen peroxide solution. In pulp and paper bleaching applications, hydrogen peroxide solutions typically have a pH between 9-13.

Selected components of exemplary polymer-stabilized aqueous hydrogen peroxide solutions are shown in the following table:

TABLE 2

| | | Standard | Pulp | | Aseptic |
|---|---|---|---|---|---|
| | Crude ca. | Grade ca. | bleaching | RO Purified | grade ca. |
| Component | 40% | 50% | ca. 2% | ca. 70% | 35% |
| (ppm) | H₂O₂ | H₂O₂ | H₂O₂ | H₂O₂ | H₂O₂ |
| Polymeric | 0.1-1500 | 0.1-1500 | 0.1-1500 | 0.2-100 | 0.1-50 |
| stabilizer | | | | | |
| Phosphate | 50-200 | 200-300 | 8-12 | 0-10 | 0-5 |
| HNO₃ | 0 | 85 | | 0-20 | 0-10 |
| NaSN | 0 | 85 | | 0-10 | 0-5 |
| Chelant | 0 | 40 | | 0 | 0-30 |
| Fe | 0.1-0.6 | 0.2-1 | | <0.5 | <0.25 |
| Cr | | | | <0.005 | <0.003 |
| Dry residue | | | | ≤16-120 | ≤8-60 |

Methods and Uses

In commercial aseptic packaging equipment which uses roll stock, the packaging material is immersed in hydrogen peroxide solution followed by heating to vaporize the peroxide before the packages are filled. Contact time with the solutions, which contain a wetting agent, is often less than 1 minute. A large amount of the sterilizing liquid is removed mechanically, e.g. by rollers or air blasts, and the remainder is generally removed by drying with hot or sterile air or radiant heat. The packaging material (i.e. plastic laminates with cardboard, films of thermoformable plastics and laminates) are taken from a reel and dipped into a bath of aqueous hydrogen peroxide. Wetting agents may be added to ensure uniform wetting of the surfaces. Excess solution is removed by squeeze rolls or air jets after removal of the material from the bath, which leaves a thin film of solution that is then dried by the application of hot air. To increase the efficacy, especially in the case of dusty or slightly soiled material, prior treatment of the material with rotating brushes, sterile compressed air jets or ultrasound applied to the bath may be added.

When sterilizing pre-formed containers, hydrogen peroxide is sprayed or atomized into the container. A measured amount of hydrogen peroxide is metered into each nozzle which delivers the solution into each container to ensure that a uniform film coats the inside surface of the package. A conventional spray may give drops of over 30 μm diameters on the surface, and 30-40% of the surface area is covered. An ultrasonic system can be used to give particle sizes of only 3 μm diameter, which will give an average surface cover of about 60%. The drying must be carried out with hot sterile air. Another method is the use of a mixture of hot air and vaporized peroxide. Sterilization by hydrogen peroxide vapor would be a cost-effective alternative as the least amount of hydrogen peroxide is used. The amount of hydrogen peroxide adsorbed on the treated surface from the vapor phase will be several orders of magnitude smaller than a liquid film. Therefore flushing the vapor-treated surface with low-temperature sterile air free of hydrogen peroxide vapors can effectively eliminate residues.

The invention provides a method of aseptic sterilization of packaging material comprising dipping the packaging material in or spraying the packaging material with the polymer-stabilized H₂O₂ solution composition of the invention. In some embodiments, the method comprises dipping the packaging material in the polymer-stabilized hydrogen peroxide solution, for example, using the technique described in the European patent application EP342485, which is incorporated herein by reference. Such processes are usually operated at a high temperature of typically 70-95° C. (e.g., 80° C.).

In some embodiments, the method comprises spraying the packaging material with the polymer-stabilized hydrogen peroxide solution. In a spray packaging process, the packaging materials are purged with hydrogen peroxide, for example, as described in the German patent application DE 19945500, EP1812084, and U.S. Pat. No. 6,786,249, which are incorporated by reference herein. The hydrogen peroxide solutions used in these processes must have a very low dry residue (e.g., ≤20 ppm) in order to prevent incrustations in the evaporator or spraying section and to avoid frequent cleaning. The dry residues can, amongst others, originate from the stabilizers present in the H₂O₂ solution. Thus, the spray technology requires a low amount of traditional stabilizer. In some embodiments, the polymer-stabilized H₂O₂ composition is sprayed as a vapor at a temperature of about 150-200° C.

In some embodiments, the hydrogen peroxide concentration does not differ from an initial value by more than 10% during 120 hours of operation according to either the dip bath or spray process.

The composition of the present invention can be used to effectively reduce the number of microbes located upon a substrate. In specific embodiments, the composition can effectively kill and/or inhibit a microorganism (e.g., virus, fungus, mold, slime mold, algae, yeast, mushroom and/or bacterium), thereby disinfecting the substrate.

In additional specific embodiments, the composition can effectively sanitize a substrate, thereby simultaneously cleaning and disinfecting the substrate. In additional specific embodiments, the composition can effectively kill or inhibit all forms of life, not just microorganisms, thereby acting as a biocide.

In specific embodiments, the composition can effectively disinfectant a substrate. In further specific embodiments, the composition can effectively disinfectant the surface of a substrate. In additional specific embodiments, the composition can effectively sterilize a substrate. In further specific embodiments, the composition can effectively sterilize the surface of a substrate.

The polymer-stabilized hydrogen peroxide solutions disclosed herein also have applications in the electronics industry as an oxidizing and/or a cleaning agent. Specific uses include use as an etchant in the production process of printed circuits boards and as an oxidizing and cleaning agent in the manufacturing process of semiconductors.

In another aspect, provided are methods of bleaching paper pulp or cellulosic fibers comprising contacting the composition of the invention with the paper pulp or the cellulosic fibers. In some embodiments, the paper pulp is a mechanical pulp, a chemical pulp, a semi-chemical pulp, a mechanical-chemical pulp, a thermomechanical pulp, or a chemi-thermomechanical pulp. In some embodiments, the paper pulp is a kraft pulp. In some embodiments, the kraft pulp is delignified kraft pulp. In some embodiments the bleaching comprises heating to 50-90° C. In some embodiments, he bleaching is under alkaline pH (e.g., 9-13).

### Examples

Stability Testing

The stability of hydrogen peroxide solutions is very important for their safe storage and use. The stability can be measured by heating a sample and measuring the peroxide remaining. This test is conducted for 16 hours at 96° C. Mixtures of peroxides with other ingredients, especially decomposition catalysts such as Fe, Cu, Mn, Pt, Os, Ag, Al, V, Ni, Cr, will decrease the stability of hydrogen peroxide solutions.

Procedure

1. Flask Preparation
1.1 Fill the flasks with 10% NaOH.
1.2 Heat the flasks at 96° C. for 60 minutes in a heating bath.
1.3 Remove the flasks from the heating bath and let them cool to room temperature.
1.4 Rinse the flasks with DIW (deionized water).
1.5 Fill the flasks with 10% HNO₃ for three hours.
1.6 Rinse the flasks thoroughly with Ultrapure water (three times).
1.7 Cover the flasks with aluminum foil.
1.8 Dry the flasks in a oven at 105° C. for one hour.
1.9 Remove the flasks from the oven and place them in a desiccator to cool to room temperature.

This cleaning must be done before each usage of the flasks. It is recommended that these flasks be dedicated to this procedure.

2. Stability Test
2.1 Analyze the sample for initial concentration of H₂O₂, by using an appropriate test method depending on whether analyzing pure solutions of H₂O₂, or the sample contains organic ingredients like surfactants, fragrances, flavors, etc.
2.2 Place 50 ml of the hydrogen peroxide being tested in a 100 ml volumetric flask prepared as at section 1. Cover the flask with a condenser cap or a centrifuge tube as an alternative.
2.3 Place the covered flasks in a 96° C. (205° F.) silicone oil or glycerin bath for 16 hours. Use an appropriate way to measure the temperature during the length of test, such as a thermocouple attached to a recorder. The flask should be immersed so that the liquid level is not above the 100 mL mark. Clamps should be used to suspend the flask in the bath or lead “donuts” should be used to prevent the flasks from overturning.
2.4 After 16 hours remove the flask from the bath and let it cool to room temperature.
2.5 Mix thoroughly the solution in the flask.
2.6 Analyze again the solution for H₂O₂ concentration using the same method as in section 2.1.

Note: For accurate results, the stability test should be conducted in duplicate.

Calculations

Decomposition [%]=(Cᵢₙᵢₜᵢₐₗ−C_{final})/Cᵢₙᵢₜᵢₐₗ×100, where Cᵢₙᵢₜᵢₐₗ=initial concentration of H₂O₂, C_{final}=concentration of H₂O₂ after heating.

In general, H₂O₂ solutions which record hot stability values of over 96.5%, (decomposition less than 3.5%), will exhibit satisfactory shelf stability for at least a 12 month period under room temperature storage.

Stability Results

Tables 3 to 6 show the % hydrogen peroxide decomposition from stability testing for aqueous hydrogen peroxide solutions containing various stabilizers and/or additives. A 50 wt % hydrogen peroxide solution containing 15 ppm nitric acid was used for the experiments of table 3. Two different 50 wt % hydrogen peroxide solutions containing 15 ppm phosphoric acid and having a reduced content of organic impurities were used for the experiments of tables 4 and 5. A 49.4 wt % hydrogen peroxide solution purified by reverse osmosis was used for the experiments of table 6. In tests conducted with a metal spike, a cocktail of metals was added corresponding to the following amounts in the hydrogen peroxide solution: 0.2 ppm iron, 0.3 ppm aluminum, 0.1 ppm chromium, and 0 ppm or 0.1 ppm nickel was added prior to the start of the stability test. Aluminum was added as a solution of 1 mg/ml of Al in 0.5N HNO₃. Chromium was added as a chromium (III) solution of 1 mg/ml of Cr in 2% HCl. Iron was added as a solution of 1 mg/ml of Fe in 2-5% HNO₃.

Tables 3 to 6 include the following abbreviations.

| | |
|---|---|
| NaHPP | Sodium hydrogen pyrophosphate |
| NaSN | Sodium stannate |
| A1000 | Acumer ™ 1000 (Dow): a polyacrylic acid with sodium |
| | hydrogen sulfite giving a pH of 3.2-4.0 and having a |
| | molecular weight of 4100-4900. |
| A445 | ACUSOL ™ 445 (Rohm and Haas): a partially neutralized |
| | homopolymer of acrylic acid giving a pH of 3.7 |
| | and having Mw of 4500. |
| A445N | ACUSOL ™ 445N (Rohm and Haas): a neutralized |
| | homopolymer of acrylic acid giving a pH of 6.9 |
| | and having Mw of 4500. |
| K-781 | CarbosperseTM K-781 Acrylate Terpolymer (Lubrizol): a |
| | partially neutralized acrylic terpolymer of acrylic acid, |
| | 2-acrylamido-2-methylpropane sulfonic acid and sulfonated |
| | styrene giving a pH of 2.2-3.2 and having a molecular weight |
| | less than 10,000. |
| A4161 | Acumer ™ 4161 (Rohm and Haas): a phosphinopolycarboxylic |
| | acid giving a pH of 3.0-3.5 and having a molecular weight |
| | of 3300-3900 measured by GPC of the acid form. |
| P9110 | Dequest ® P9110 (Italmatch): a phosphinopolycarboxylic |
| | acid giving a pH of 3.5-5 and having Mw of 4500-5500 g/mol. |
| P9500 | Dequest ® P9500 (Italmatch): a partially neutralized |
| | terpolymer of acrylic acid, |
| | 2-acrylamido-2-methylpropanesulfonic |
| | acid and sodium phosphinite giving a pH of 1.5-3.0. |
| X | Metal spike providing 0.1 ppm Nickel |
| XX | Metal spike providing no Nickel |

TABLE 3
Stabilizer added

| NaHPP | NaSN | A1000 | DTPMP | ATMP | Metal | Decomposition |
|---|---|---|---|---|---|---|
| (ppm) | (ppm) | (ppm) | (ppm) | (ppm) | Spike | result |
| 2.5 | 5 | 0 | 0 | 0 | | 0.45% |
| 2.5 | 5 | 2.5 | 0 | 0 | | 0.77% |
| 2.5 | 5 | 2.5 | 2.5 | 0 | | 1.02% |
| 2.5 | 5 | 2.5 | 0 | 2.5 | | 1.08% |
| 2.5 | 5 | 0 | 0 | 0 | X | 9.30% |
| 2.5 | 5 | 2.5 | 0 | 0 | X | 31.40% |
| 2.5 | 5 | 2.5 | 2.5 | 0 | X | 9.20% |
| 2.5 | 5 | 5 | 2.5 | 0 | X | 7.20% |

TABLE 4
Stabilizer added

| | | | | | | | | De- |
|---|---|---|---|---|---|---|---|---|
| | | | | | | | | com- |
| | | | | | | | | posi- |
| NaHPP | NaSN | A1000 | A445 | DTPMP | ATMP | K-781 | Metal | tion |
| (ppm) | (ppm) | (ppm) | (ppm) | (ppm) | (ppm) | (ppm) | Spike | result |
| 2.5 | 5 | 0 | | 0 | 0 | 0 | | 1.61% |
| 2.5 | 5 | 2.5 | | 0 | 0 | 0 | | 2.54% |
| 2.5 | 5 | 2.5 | | 2.5 | 0 | 0 | | 0.85% |
| 2.5 | 2.5 | 2.5 | | 0 | 2.5 | 0 | | 1.97% |
| 2.5 | 2.5 | 0 | | 0 | 0 | 10 | | 0.91% |
| 2.5 | 5 | 0 | | 0 | 0 | 0 | X | 3.90% |
| 2.5 | 5 | 2.5 | | 2.5 | 0 | 0 | X | 5.40% |
| 2.5 | 5 | 5 | | 2.5 | 0 | 0 | X | 5.60% |
| 2.5 | 5 | 2.5 | | 5 | 0 | 0 | X | 7.60% |
| 2.5 | 5 | 0 | | 5 | 0 | 0 | XX | 7.06% |
| 2.5 | 5 | 0 | | 10 | 0 | 0 | XX | 1.67% |
| 2.5 | 5 | 5 | | 5 | 0 | 0 | XX | 2.96% |
| 2.5 | 5 | 5 | | 2.5 | 0 | 0 | XX | 5.60% |
| 2.5 | 5 | 0 | 5 | 5 | 0 | 0 | XX | 2.70% |
| 2.5 | 5 | 0 | 10 | 0 | 0 | 0 | XX | 5.10% |

TABLE 5
Stabilizer added

| NaHPP | NaSN | A445N | A4161 | Metal | Decomposition |
|---|---|---|---|---|---|
| (ppm) | (ppm) | (ppm) | (ppm) | Spike | result |
| 2.5 | 5 | 50 | 0 | X | 3.62% |
| 2.5 | 5 | 25 | 0 | X | 4.16% |
| 2.5 | 5 | 12.5 | 0 | X | 4.42% |
| 2.5 | 5 | 0 | 50 | X | 2.88% |
| 2.5 | 5 | 0 | 25 | X | 1.88% |
| 2.5 | 5 | 0 | 12.5 | X | 1.88% |

TABLE 6
Stabilizer added

| | | | | | | Decom- |
|---|---|---|---|---|---|---|
| NaHPP | NaSN | A4161 | P9110 | P9500 | K-781 | position |
| (ppm) | (ppm) | (ppm) | (ppm) | (ppm) | (ppm) | result |
| 0 | 0 | 0 | 0 | 0 | 0 | 57.3% |
| 0 | 0 | 10 | 0 | 0 | 0 | 1.4% |
| 0 | 0 | 20 | 0 | 0 | 0 | 1.3% |
| 0 | 0 | 100 | 0 | 0 | 0 | 0.5% |
| 0 | 0 | 200 | 0 | 0 | 0 | 1.1% |
| 0 | 0 | 0 | 20 | 0 | 0 | 1.7% |
| 0 | 0 | 0 | 0 | 20 | 0 | 1.8% |
| 0 | 0 | 0 | 0 | 0 | 100 | 0.8% |

It is understood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, compositions, formulations, or methods of use of the invention, may be made without departing from the spirit and scope thereof.

For reasons of completeness, various aspects of the invention are set out in the following numbered clauses:

Clause 1. An aqueous composition comprising

hydrogen peroxide; and

one or more polymeric stabilizers selected from
a) a phosphino polycarboxylic acid, or salt thereof, the phosphino polycarboxylic acid having a molecular weight of 1500 to 10,000 g/mol; and
b) a polymer, or salt thereof, with molecular weight of 3000 to 15.000 g/mol, the polymer being derived from a plurality of monomer units of each of

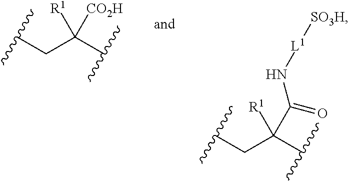

and optionally

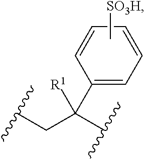

wherein R¹, at each occurrence, is independently hydrogen or C₁₋₄alkyl and L¹ is C₂₋₆alkylene.

Clause 2. The composition of clause 1, wherein the one or more polymeric stabilizers is selected from the phosphino polycarboxylic acid, or a salt thereof.

Clause 3. The composition of clause 2, wherein the phosphino polycarboxylic acid has formula (I):

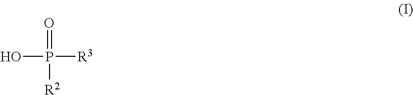

wherein

R² is

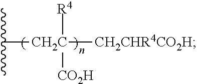

R³ is

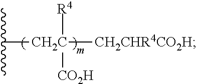

R⁴, at each occurrence, is independently hydrogen or C₁₋₄alkyl; and m and n are each independently an integer, where m+n is an integer from 30 to 60.

Clause 4. The composition of clause 3, wherein R⁴ is hydrogen.

Clause 5. The composition of any of clauses 2-4, wherein the phosphino polycarboxylic acid has a molecular weight of 3300-3900 g/mol.

Clause 6. The composition of clause 1, wherein the one or more polymeric stabilizers is selected from a polymer, or salt thereof, with molecular weight of 3000 to 15,000 g/mol, the polymer being derived from a plurality of monomer units of each of

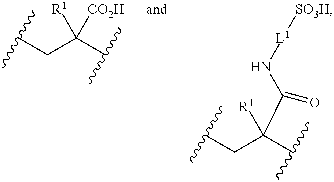

wherein R¹, at each occurrence, is independently hydrogen or C₁₋₄alkyl and L¹ is C₂₋₆alkylene.

Clause 7. The composition of clause 6, wherein the polymer is derived from a plurality of monomer units of each of

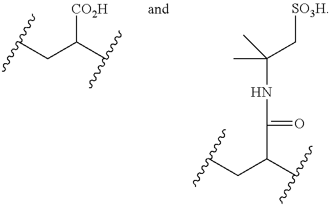

Clause 8. The composition of clause 1, wherein the one or more polymeric stabilizers is selected from a polymer, or salt thereof, with molecular weight of 3000 to 15,000 g/mol, the polymer being derived from a plurality of monomer units of each of

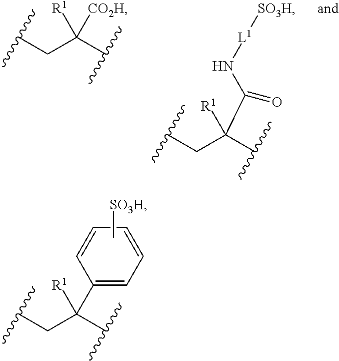

wherein R′, at each occurrence, is independently hydrogen or C₁₋₄alkyl and L¹ is C₂₋₆alkylene.

Clause 9. The composition of clause 8, wherein the polymer is derived from a plurality of monomer units of each of

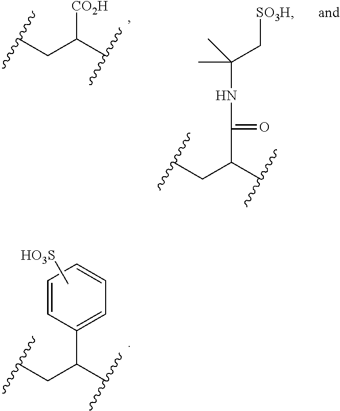

Clause 10. The composition of any of clauses 1-9, comprising from 5 to 80% by weight hydrogen peroxide and from 0.1 to 1500 ppm of the one or more polymeric stabilizers.

Clause 11. The composition of any of clauses 1-10, wherein a 35 weight % hydrogen peroxide solution comprises ≤60 ppm of the one or more polymeric stabilizers.

Clause 12. The composition of any of clauses 1-11 wherein the composition is substantially free of a stannate and/or chelating substance other than the one or more polymeric stabilizers.

Clause 13. The composition of any of clauses 1-12 having a phosphorus content expressed as PO₄³⁻ of ≤10 ppm.

Clause 14. A method of aseptic sterilization of packaging material comprising dipping the packaging material in or spraying the packaging material with the composition of any of clauses 1-13.

Clause 15. The method of clause 14 comprising dipping the packaging material in the composition of any of clauses 1-13 at 70-95° C.

Clause 16. The method of clause 14, comprising spraying the packaging material with the composition of any of clauses 1-13, the composition being sprayed as a vapor at a temperature of about 150-200° C.

Clause 17. A method of bleaching paper pulp or cellulosic fibers comprising contacting the composition of any of clauses 1-13 with the paper pulp or the cellulosic fibers.

Clause 18. The method of clause 17 comprising bleaching kraft pulp.

Clause 19. The method of clause 17 comprising bleaching a chemi-thermomechanical pulp.

1. An aqueous composition comprising
hydrogen peroxide; and
one or more polymeric stabilizers selected from
a) a phosphino polycarboxylic acid, or salt thereof, the phosphino polycarboxylic acid having a molecular weight of 1500 to 10,000 g/mol; and
b) a polymer, or salt thereof, with molecular weight of 3000 to 15,000 g/mol, the polymer being derived from a plurality of monomer units of each of 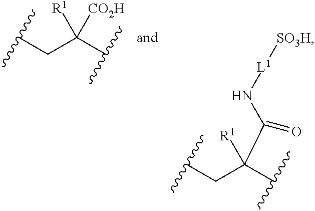
and optionally 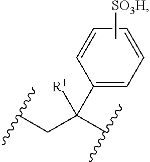
wherein R¹, at each occurrence, is independently hydrogen or C₁₋₄alkyl and L¹ is C₂₋₆alkylene.

2. The composition of claim 1, wherein the one or more polymeric stabilizers is selected from the phosphino polycarboxylic acid, or a salt thereof.

3. The composition of claim 2, wherein the phosphino polycarboxylic acid has formula (I): 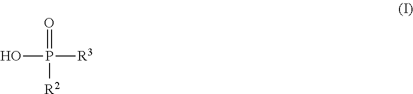
wherein
R² 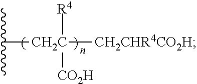
R³ is 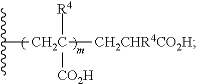
R⁴, at each occurrence, is independently hydrogen or C₁₋₄alkyl; and
m and n are each independently an integer, where m+n is an integer from 30 to 60.

4. The composition of claim 3, wherein R⁴ is hydrogen.

5. The composition of claim 2, wherein the phosphino polycarboxylic acid has a molecular weight of 3300-3900 g/mol.

6. The composition of claim 1, wherein the one or more polymeric stabilizers is selected from a polymer, or salt thereof, with molecular weight of 3000 to 15,000 g/mol, the polymer being derived from a plurality of monomer units of each of 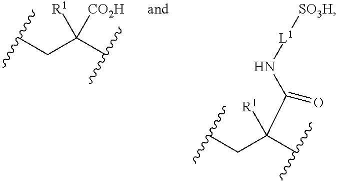
wherein R¹, at each occurrence, is independently hydrogen or C₁₋₄alkyl and L¹ is C₂₋₆alkylene.

7. The composition of claim 6, wherein the polymer is derived from a plurality of monomer units of each of

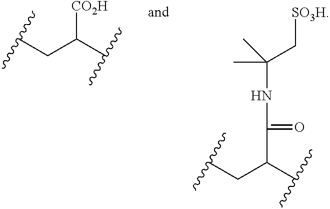

8. The composition of claim 1, wherein the one or more polymeric stabilizers is selected from a polymer, or salt thereof, with molecular weight of 3000 to 15,000 g/mol, the polymer being derived from a plurality of monomer units of each of 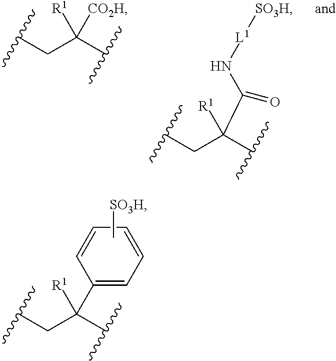
wherein R¹, at each occurrence, is independently hydrogen or C₁₋₄alkyl and L¹ is C₂₋₆alkylene.

9. The composition of claim 8, wherein the polymer is derived from a plurality of monomer units of each of

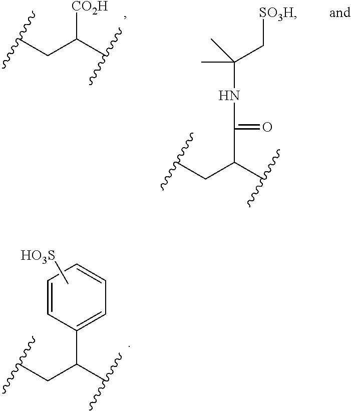

10. The composition of claim 1, comprising from 5 to 80% by weight hydrogen peroxide and from 0.1 to 1500 ppm of the one or more polymeric stabilizers.

11. The composition of claim 1, wherein a 35 weight % hydrogen peroxide solution comprises ≤60 ppm of the one or more polymeric stabilizers.

12. The composition of claim 1 wherein the composition is substantially free of a stannate and/or chelating substance other than the one or more polymeric stabilizers.

13. The composition of claim 1 having a phosphorus content expressed as PO₄³⁻ of ≤10 ppm.

14. A method of aseptic sterilization of packaging material comprising dipping the packaging material in or spraying the packaging material with the composition of claim 1.

15. The method of claim 14, wherein the packaging material is dipped in the composition at 70-95° C.

16. The method of claim 14, wherein the composition is sprayed on the packaging material as a vapor at a temperature of about 150-200° C.

17. A method of bleaching paper pulp or cellulosic fibers comprising contacting the composition of claim 1 with the paper pulp or the cellulosic fibers.

18. The method of claim 17 comprising bleaching kraft pulp.

19. The method of claim 17 comprising bleaching a chemi-thermomechanical pulp.

20. The composition of claim 2, comprising from 5 to 80% by weight hydrogen peroxide and from 0.1 to 1500 ppm of the one or more polymeric stabilizers.
